(19) United States
(12) Patent Application Publication
(10) Pub. No.: US 2022/0111107 A1
(43) Pub. Date: Apr. 14, 2022

(54) LIGHT STERILIZING APPARATUS

(71) Applicants: ZHUHAI MEASURE SCIENCE AND TECHNOLOGY CO., LTD, Zhuhai, Guangdong (CN); MEASURE INC., Broomfield, CO (US)

(72) Inventors: Panfeng Xiang, Zhuhai, Guangdong (CN); Bo Zou, Zhuhai, Guangdong (CN)

(21) Appl. No.: 17/267,198

(22) PCT Filed: Jul. 10, 2020
(86) PCT No.: PCT/CN2020/101187

§ 371 Date: Feb. 9, 2021

(30) Foreign Application Priority Data
Mar. 20, 2020 (CN) 202010205379.5
Jun. 24, 2020 (CN) 202010595612.5

(51) Int. Cl. A61L 9/20; F24F 8/22; B60H 3/00

(52) U.S. Cl. CPC A61L 9/20; B60H 3/0078; F24F 8/22

(57) ABSTRACT

A light sterilizing apparatus includes a light source mechanism and a flow carrier, wherein the light source mechanism is positioned outside the flow carrier, the light source mechanism emits sterilizing light with a wavelength of 200-280 nm, the light emitted by the light source mechanism passes through a light transmission part of the flow carrier and enters a channel part of the light source mechanism, and a channel in the channel part is a winding channel. Configuring the channel as a winding channel prolongs the duration of air flowing in the flow carrier, and thus renders a longer sterilization process, which guarantees the sterilization effect and effectively reduces the size of the light source mechanism and the flow carrier; as a result, the light sterilizing apparatus can be suitable for various scenarios and wide application.

### TECHNICAL FIELD

The invention relates to the technical field of sterilization, more particularly, to a light sterilizing apparatus.

### BACKGROUND ART

The light sterilizing apparatus in the prior art mostly adopts a straight-through pipeline, with air entering from an inlet, subjected to sterilization by the light source mechanism, and discharged from an outlet of the straight-through pipeline. Because the air in the straight-through pipeline flows in a single direction, the pipeline and the light source mechanism have to be designed to be large enough to achieve the expected sterilization effect, as a result, the light sterilizing apparatus is limited in use and difficult to widely apply.

### SUMMARY OF THE INVENTION

It is a major object of the invention to provide a light sterilizing apparatus suitable for wide application.

To achieve the major object, the light sterilizing apparatus provided herein includes a light source mechanism and a flow carrier, the light source mechanism is positioned outside the flow carrier, and the light source mechanism emits sterilizing light with a wavelength of 200-280 nm; the light sterilizing apparatus further includes a holder and a reflector, the light source mechanism is provided on the outer sides of two ends of the holder, respectively, sterilizing light emitted by the light source mechanism sequentially passes through an end plate of the holder, a light passing hole of the reflector and a light transmission part of the flow carrier, and enters a channel part of the flow carrier; two flow carriers are provided in the holder, a partition plate is provided between the two flow carriers, a central hole of the partition plate is communicated with an outlet of the channel of one flow carrier and a inlet of the channel of the other flow carrier, and the reflector is disposed between each flow carrier and an inner end surface of the holder; an inlet and an outlet are provided on a side wall of the holder, and the inlet and the outlet are communicated with the channel part of the flow carrier, respectively; and the channel in the channel part is a winding channel.

According to the technical solution, air enters the flow carrier from the inlet and leaves the light sterilizing apparatus from the outlet, configuring the channel as a winding channel prolongs the duration of air flowing in the flow carrier, and thus renders a longer sterilization process, which guarantees the sterilization effect and effectively reduces the size of the light source mechanism and the flow carrier; as a result, the light sterilizing apparatus can be suitable for various scenarios and wide application.

Preferably, the holder includes a holder case and a fastener which are detachably fixed and connected with each other, the end plate is provided at one end of the holder case, and the end plate and the fastener are both provided with coaxially arranged through holes.

Further, the light source mechanism includes a circuit part, and the circuit part is provided with a plurality of radiating fins arranged uniformly along a circumferential direction around an optical axis of the sterilizing light; the flow carrier is a winding through pipe with a part of the pipe close to the light source mechanism being the light transmission part and a part of the pipe away from the light source mechanism being the channel part.

Further, the through pipe is at least one of a round through pipe or a square through pipe.

Preferably, the channel part is cylindrical, a top of the channel is open, and the channel is wound in the channel part.

Further, the light transmission part is detachably provided and fixed at the open top, and the light transmission part is made of glass or plastic material.

Preferably, the sterilizing light emitted by the light source mechanism is an ultraviolet laser with a wavelength of 200-280 nm, the light source mechanism includes a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

Further, the fixture is a hollow cylinder.

Preferably, the sterilizing light emitted by the light source mechanism is ultraviolet LED light with a wavelength of 200-280 nm, the light source mechanism includes a positioning frame, an LED epitaxial wafer and a second collimating lens, wherein the LED epitaxial wafer is provided in the positioning frame, and the second collimating lens is clamped in the positioning frame.

Further, the positioning frame is a square cylinder having a cavity formed therein, the LED epitaxial wafer is positioned in the cavity, and a part of the second collimating lens is exposed out of the positioning frame.

### BRIEF DESCRIPTION OF THE DRAWINGS

The invention is further illustrated with reference to the accompanying drawings, but embodiments disclosed in the drawings are not to be construed as limiting the invention. Other drawings on such a basis may be devised by those skilled in the art without involving any inventive effort.

FIG. 1 is a sectional view showing a structure of Embodiment 1 of a light sterilizing apparatus according to the present invention.

FIG. 2 is a sectional view showing a structure of a light source mechanism of Embodiment 1 of the light sterilizing apparatus according to the present invention, the light source mechanism emitting an ultraviolet laser with a wavelength of 200 to 280 nm as sterilizing light.

FIG. 3 is a sectional view showing a structure of a light source mechanism of Embodiment 1 of the light sterilizing apparatus according to the present invention, the light source mechanism emitting ultraviolet LED light with a wavelength of 200 to 280 nm as sterilizing light.

FIG. 4 is a structural view of a flow carrier of Embodiment 1 of the light sterilizing apparatus according to the present invention.

FIG. 5 is a schematic flow diagram of a fluid of Embodiment 1 of the light sterilizing apparatus according to the present invention.

FIG. 6 is a partially exploded sectional view of a structure of a mask to which the light sterilizing apparatus according to the present invention is applied.

FIG. 7 is a schematic view of Embodiment 1 of the light sterilizing apparatus according to the present invention when applied to an automobile.

FIG. 8 is a schematic view of Embodiment 1 of the light sterilizing apparatus according to the present invention when applied to a house.

FIG. 9 is a schematic view of Embodiment 1 of the light sterilizing apparatus according to the present invention when applied to goggles.

FIG. 10 is a structural diagram of Embodiment 2 of the flow carrier of the light sterilizing apparatus according to the present invention.

FIG. 11 is a structural diagram of Embodiment 3 of the flow carrier of the light sterilizing apparatus according to the present invention.

FIG. 12 is a schematic view showing a structure of Embodiment 4 of the flow carrier of the light sterilizing apparatus according to the present invention.

FIG. 13 is a schematic view showing a structure of Embodiment 5 of the flow carrier of the light sterilizing apparatus according to the present invention.

FIG. 14 is a modified embodiment based on Embodiment 5.

FIG. 15 is another modified embodiment based on Embodiment 5.

### DETAILED DESCRIPTION OF THE INVENTION

In order that the object, aspects, and advantages of the present invention will become more apparent, a more particular description of the invention will be rendered by reference to the accompanying drawings and examples. It should be understood that the specific embodiments described herein are merely illustrative of the invention and are not intended to be limiting.

### Embodiment 1

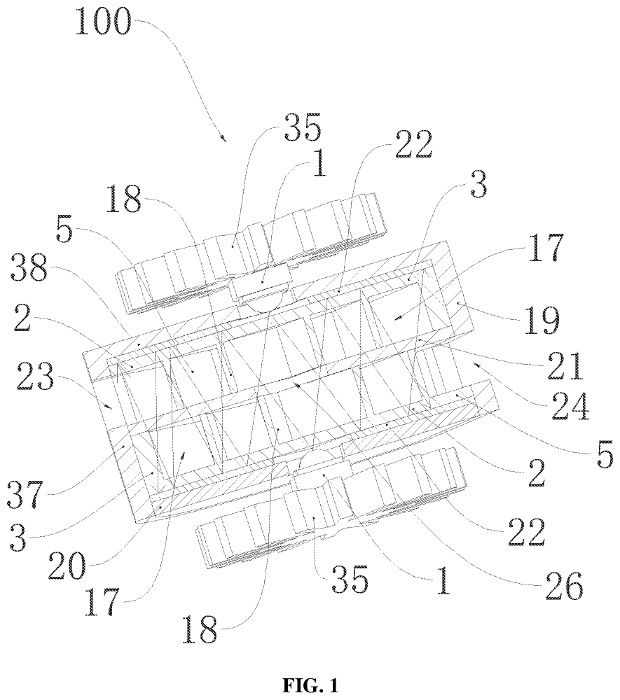

Referring to FIG. 1, the light sterilizing apparatus 100 includes a light source mechanism 1 and a flow carrier 2, wherein the light source mechanism 1 is positioned outside the flow carrier 2, the light source mechanism 1 emits sterilizing light with a wavelength of 200-280 nm, the light emitted by the light source mechanism 1 passes through a light transmission part 3 of the flow carrier 2 and enters a channel part 5 of the flow carrier 2, and a channel 17 in the channel part 5 is a winding channel. The light transmission part is a glass or plastic piece, a first section of the channel 17 is in a plane parallel to an optical axis of the sterilizing light, and the first section of the channel 17 is in at least one of a circular shape, a semicircular shape, an elliptical shape or a polygonal shape. The winding channel indicates that the direction of a fluid flowing in the channel is variable.

The holder 19 includes a holder case 37 and a fastener 20 which are detachably fixed and connected with each other, and the holder case 37 and the fastener 20 are in threaded connection. In this embodiment, the holder case 37 is provided with an end plate 38 at one end thereof, the end plate 38 and the fastener 20 are both provided with coaxially arranged through holes, and the end plate 38 and the fastener 20 are oppositely arranged along the optical axis of sterilizing light. The end plate 38 may also be provided at both ends of the holder case 37 to replace the fastener 20.

The sterilizing light emitted by one of the two light source mechanisms 1 sequentially passes through the end plate 38 of the holder 19, a light passing hole of a reflector 22 and the light transmission part 3 of the flow carrier 2, and enters the channel part 5 of the flow carrier 2, while the sterilizing light emitted by the other light source mechanism 1 can pass through the fastener 20, the holder 19 and the light transmission part 3 of the flow carrier 2, and enter the channel part 5 of the flow carrier 2. Two flow carriers 2 are provided in the holder 19. Each flow carrier 2 is provided with a light source mechanism 1, a part, close to the light source mechanism 1 provided therefor, of the flow carrier 2 along the optical axis of sterilizing light is the light transmission part 3, and a part, away from the light source mechanism 1 provided therefor, of the flow carrier 2 along the optical axis of sterilizing light is the channel part 5. A partition plate 21 is provided between the two carriers 2, a central hole 26 of the partition plate 21 is communicated with an outlet of the channel of one carrier 2 and an inlet of the channel of the other carrier 2, the reflector 22 is disposed between each carrier 2 and an inner end surface of the holder 19, an inlet 23 and an outlet 24 are provided on a side wall of the holder 19, and the inlet and the outlet are communicated with the channel part 5 of either carrier 2, respectively. The reflector 22 reflects the sterilizing light for several times upon the arrival of the sterilizing light at the reflector 22, thereby increasing a light path of the sterilizing light in the holder 19 and improving the sterilizing effect. The light source mechanism 1 is in fixed electrical connection with a connector 35 which is connected to a power supply.

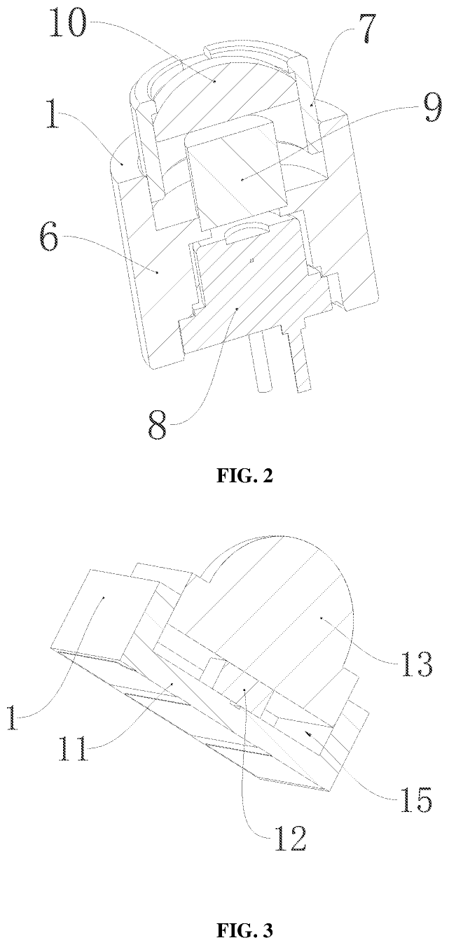

Referring to FIGS. 1 and 2, the sterilizing light emitted by the light source mechanism 1 is an ultraviolet laser with a wavelength of 200-280 nm, the light source mechanism 1 includes a fixture 6, a positioning sleeve 7, a light-emitting element 8, a conversion crystal 9 and a first collimating lens 10, wherein the light-emitting element 8 is inserted into the fixture 6, and the fixture 6 is sleeved around an outer wall of the positioning sleeve 7. The positioning sleeve 7 is sleeved outside the first collimating lens 10, and the conversion crystal 9 is positioned in the fixture 6 which is a hollow cylinder.

Referring to FIGS. 1 and 3, the light emitted by the light source mechanism 1 is an ultraviolet LED light with a wavelength of 200-280 nm, the light source mechanism 1 includes a positioning frame 11, an LED epitaxial wafer 12 and a second collimating lens 13, wherein the LED epitaxial wafer 12 is provided in the positioning frame 11, and the second collimating lens 13 is clamped in the positioning frame 11. The positioning frame 11 is a square cylinder having a cavity 15 therein, the LED epitaxial wafer 12 is positioned in the cavity 15, and a part of the second collimating lens 13 is exposed out of the positioning frame 11.

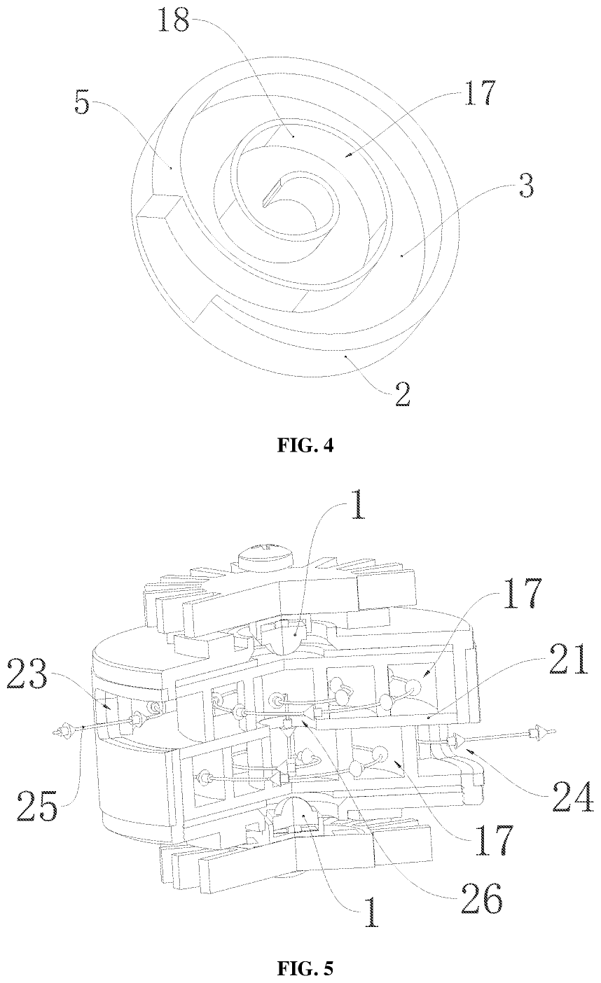

Referring to FIGS. 1, 4 and 5, a winding wall 18 is provided in the channel part 5 to form the winding channel 17 in which a fluid 25 flows in a winding direction, the direction of the fluid is indicated by a tangent at a position where the fluid is present, and the direction of the fluid flowing in the channel 17 is variable.

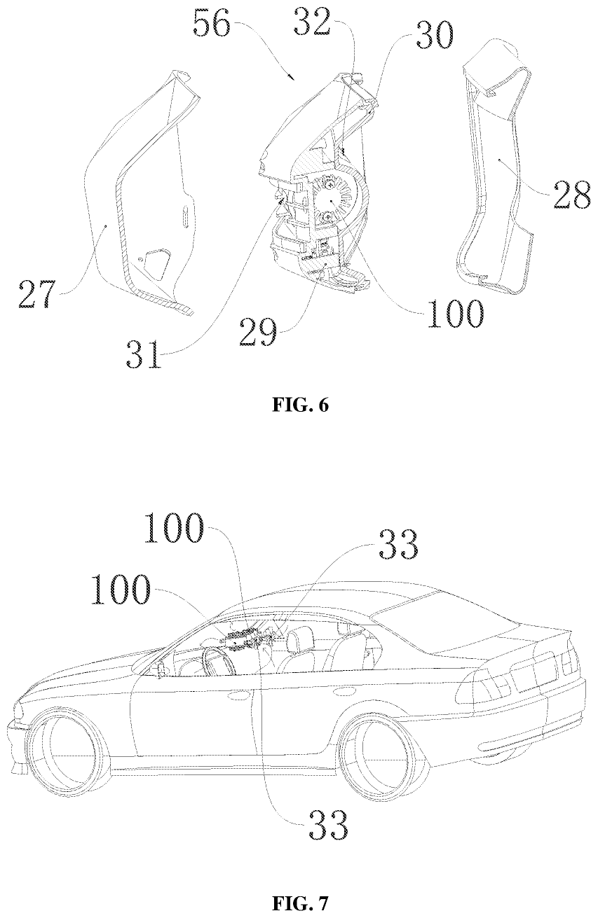

Referring to FIG. 6, a mask 26 employs the light sterilizing apparatus 100, the mask 26 includes an outer layer 27, a silica gel inner layer 28, a first cover 29 and a second cover 30, the silica gel inner layer 28 conforms to the face of a person, an opening 31 in the first cover 29 communicates with the inlet 23 of the light sterilizing apparatus 100, and an opening 32 in the second cover 30 communicates with the outlet 24 of the light sterilizing apparatus 100. Gas, which is a fluid, enters the mask 26 from the space between the outer layer 27 and the silica gel layer 28, and then enters the light sterilizing apparatus 100 from the opening 31 in the first cover 29; the sterilized gas is discharged from the opening 32 for the person to breathe in. The power supply is a lithium battery, the connector 35 is electrically connected to a control panel, and the lithium battery and the control panel are fixed between the first cover 29 and the second cover 30.

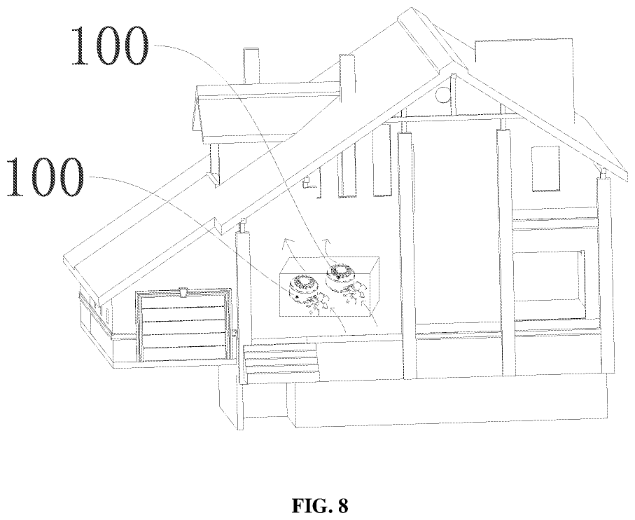

Referring to FIGS. 7 and 8, the light sterilizing apparatus 100 is placed in an enclosure provided with a gas inlet and a gas outlet, a fan 33 is provided at the gas inlet for drawing air into a gas channel connecting the fan 33 and the light sterilizing apparatus 100; the gas enters the light sterilizing apparatus 100 along the gas channel and then is sterilized and discharged from the gas outlet, to achieve sterilization of gases in an automobile or a house. The arrows shown in FIGS. 7 and 8 indicate the direction of the gas flow, a battery and a control panel are provided in the enclosure, and the connector 35 is electrically connected to the control panel.

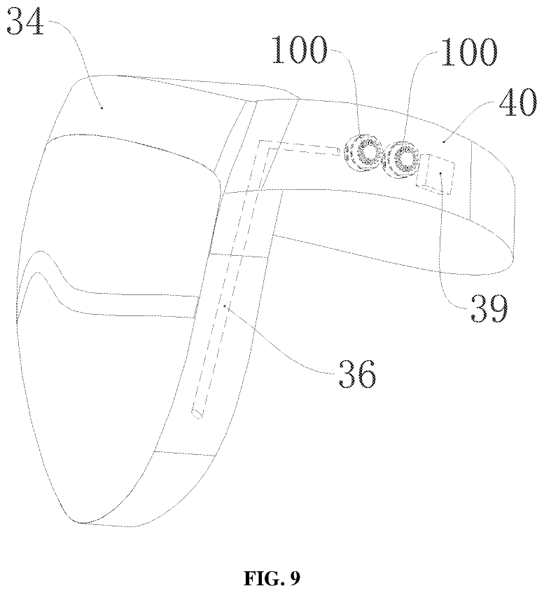

Referring to FIG. 9, the light sterilizing apparatus 100 is applied to goggles 34, the light sterilizing apparatus 100 is positioned in a head frame 40 of the goggles, the head frame 40 is integral to the overall frame of the goggles, a gas channel 36 is provided in the frame, and the gas sterilized by the light sterilizing apparatus 100 enters the human body through the gas channel 36. A battery and a control panel 39 are provided in the head frame 40, and the connector 35 is electrically connected to the control panel 39.

### Embodiment 2

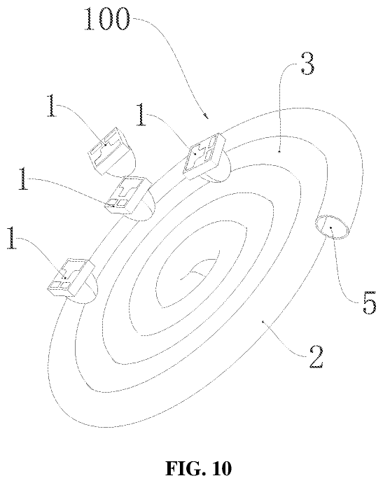

Referring to FIG. 10, the flow carrier 2 is a winding through pipe, the flow carrier 2 is made of glass or transparent plastic material, the flow carrier 2 is integrally formed, and is wound into one layer along the optical axis of sterilizing light. The fluid flows in the channel in a winding direction, the direction of the fluid is indicated by a tangent at a position where the fluid is present, and the direction of the fluid flowing in the channel is variable. The light sterilizing apparatus of Embodiment 2 may also be applied to the mask, automobile, house or goggles of Embodiment 1.

### Embodiment 3

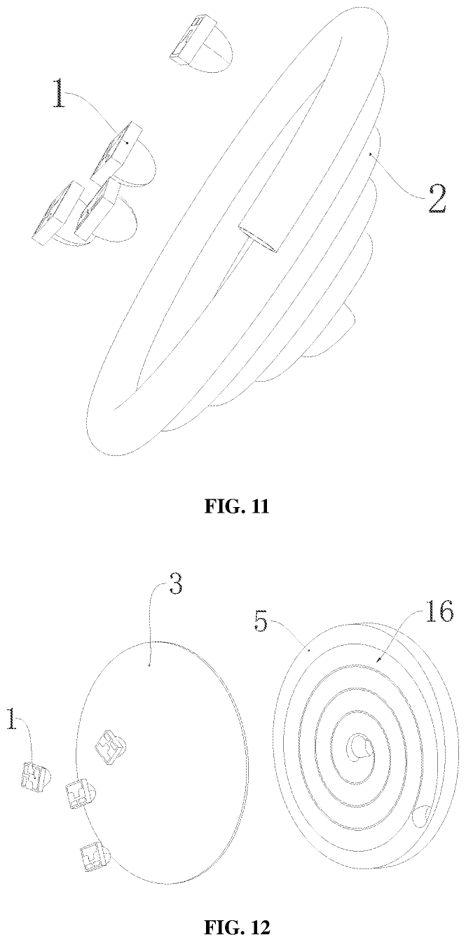

Referring to FIG. 11, the flow carrier 2 is a winding through pipe, the flow carrier 2 is integrally formed and is made of glass or transparent plastic material, and the flow carrier 2 is wound into multiple layers along the optical axis of sterilizing light. The fluid flows in the channel in a winding direction, the direction of the fluid is indicated by a tangent at a position where the fluid is present, and the direction of the fluid flowing in the channel is variable. Other configurations are the same as those Embodiment 1 and will not be described in detail herein. The light sterilizing apparatus of Embodiment 3 may also be applied to the mask, automobile, house or goggles of Embodiment 1.

### Embodiment 4

Referring to FIG. 12, the channel part 5 is a cylinder, a top of the channel 16 is open, the channel 16 is wound in the channel part 5, and the channel part 5 is of an opaque structure. The fluid flows in the channel in a winding direction, the direction of the fluid is indicated by a tangent at a position where the fluid is present, and the direction of the fluid flowing in the channel is variable.

The light transmission part 3 is detachably provided and fixed at the open top, the light transmission part 3 is a round plate and bonds to the open top. Other configurations are the same as those of Embodiment 1 and will not be described in detail herein. The light sterilizing apparatus of Embodiment 4 may also be applied to the mask, automobile, house or goggles of Embodiment 1.

### Embodiment 5

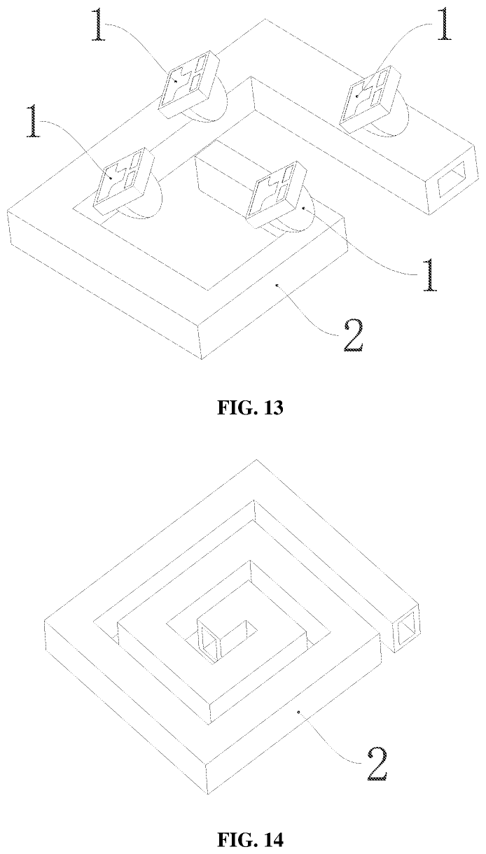

Referring to FIG. 13, the flow carrier 2 is a winding through pipe, the flow carrier 2 includes a plurality of sections of the channel, the fluid flows in the channel in a winding direction, each section of channel extends along the direction of the fluid flowing in the section of the channel, adjacent sections extend in different directions, and the direction of the fluid flowing in the channel is variable. Other configurations are the same as those of Embodiment 1 and will not be described in detail herein. The light sterilizing apparatus of Embodiment 5 may also be applied to the mask, automobile, house or goggles of Embodiment 1.

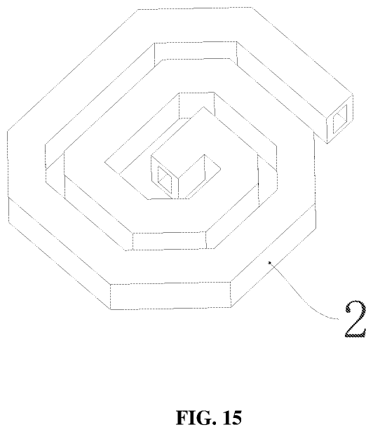

Referring to FIGS. 14 and 15, the structure of the flow carrier 2 is shown as a modified embodiment base on Embodiment 5.

Therefore, compared with the straight-through pipeline with a single direction for the flowing fluid in the prior art, configuring the channel as a winding channel enables a variable direction for the air as the fluid flowing in the winding channel, prolongs the duration of air flowing in the flow carrier 2, and thus renders a longer sterilization process, which guarantees the sterilization effect and effectively reduces the size of the light source mechanism 1 and the flow carrier 2; as a result, the light sterilizing apparatus 100 can be suitable for various scenarios and wide application.

The light source mechanism 1 can be clamped, bonded or screwed on a wall, at an air inlet of a room, at an air outlet of a vehicle-mounted air conditioner and the like, to realize the application of the light sterilizing apparatus 100 in different scenarios.

Finally, it should be noted that the present invention is not limited to the above-described embodiments, and that the above-described embodiments are merely preferred embodiments of the present invention and are not intended to limit the invention. The intention is intended to cover all modifications, equivalents, improvements, and the made without departing the spirit and scope of the invention.

1. A light sterilizing apparatus, wherein:
the light sterilizing apparatus comprises a light source mechanism and a flow carrier, the light source mechanism is positioned outside the flow carrier, and the light source mechanism emits sterilizing light with a wavelength of 200-280 nm;
the light sterilizing apparatus further comprises a holder and a reflector, the light source mechanism is provided on the outer sides of two ends of the holder, respectively, sterilizing light emitted by the light source mechanism sequentially passes through an end plate of the holder, a light passing hole of the reflector and a light transmission part of the flow carrier, and enters a channel part of the flow carrier;
two flow carriers are provided in the holder, a partition plate is provided between the two flow carriers, a central hole of the partition plate is communicated with an outlet of the channel of one flow carrier and an inlet of the channel of the other flow carrier, and the reflector is disposed between each flow carrier and an inner end surface of the holder;
an inlet and an outlet are provided on a side wall of the holder, and the inlet and the outlet are communicated with the channel part of the flow carrier, respectively; and
the channel in the channel part is a winding channel.

2. The light sterilizing apparatus according to claim 1, wherein
the holder comprises a holder case and a fastener which are detachably fixed and connected with each other, the end plate is provided at one end of the holder case, and the end plate and the fastener are both provided with coaxially arranged through holes.

3. The light sterilizing apparatus according to claim 2, wherein:
the light source mechanism comprises a circuit part, and the circuit part is provided with a plurality of radiating fins arranged uniformly along a circumferential direction around an optical axis of the sterilizing light;
the flow carrier is a winding through pipe with a part of the pipe close to the light source mechanism being the light transmission part and a part of the pipe away from the light source mechanism being the channel part.

4. The light sterilizing apparatus according to claim 3, wherein:
the through pipe is at least one of a round through pipe or a square through pipe.

5. The light sterilizing apparatus according to claim 2, wherein:
the channel part is a cylinder, a top of the channel is open, and the channel is wound in the channel part.

6. The light sterilizing apparatus according to claim 5, wherein:
the light transmission part is detachably provided and fixed at the open top, and the light transmission part is made of glass or plastic material.

7. The light sterilizing apparatus according to claim 1, wherein:
the sterilizing light emitted by the light source mechanism is an ultraviolet laser with a wavelength of 200-280 nm, the light source mechanism comprises a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

8. The light sterilizing apparatus according to claim 7, wherein:
the fixture is a hollow cylinder.

9. The light sterilizing apparatus according to claim 1, wherein:
the sterilizing light emitted by the light source mechanism is ultraviolet LED light with a wavelength of 200-280 nm, the light source mechanism comprises a positioning frame, an LED epitaxial wafer and a second collimating lens, wherein the LED epitaxial wafer is provided in the positioning frame, and the second collimating lens is clamped in the positioning frame.

10. The light sterilizing apparatus according to claim 9, wherein:
the positioning frame is a square cylinder having a cavity formed therein, the LED epitaxial wafer is positioned in the cavity, and a part of the second collimating lens is exposed out of the positioning frame.

11. The light sterilizing apparatus according to claim 2, wherein:
the sterilizing light emitted by the light source mechanism is an ultraviolet laser with a wavelength of 200-280 nm, the light source mechanism comprises a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

12. The light sterilizing apparatus according to claim 3, wherein:
the sterilizing light emitted by the light source mechanism is an ultraviolet laser with a wavelength of 200-280 nm, the light source mechanism comprises a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

13. The light sterilizing apparatus according to claim 4, wherein:
the sterilizing light emitted by the light source mechanism is an ultraviolet laser with a wavelength of 200-280 nm, the light source mechanism comprises a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

14. The light sterilizing apparatus according to claim 5, wherein:
the sterilizing light emitted by the light source mechanism is an ultraviolet laser with a wavelength of 200-280 nm, the light source mechanism comprises a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

15. The light sterilizing apparatus according to claim 6, wherein:
the sterilizing light emitted by the light source mechanism is an ultraviolet laser with a wavelength of 200-280 nm, the light source mechanism comprises a fixture, a positioning sleeve, a light-emitting element, a conversion crystal and a first collimating lens, wherein the light-emitting element is inserted into the fixture, the fixture is sleeved on an outer wall of the positioning sleeve, the positioning sleeve is sleeved on the first collimating lens, and the conversion crystal is positioned in the fixture.

16. The light sterilizing apparatus according to claim 2, wherein:
the sterilizing light emitted by the light source mechanism is ultraviolet LED light with a wavelength of 200-280 nm, the light source mechanism comprises a positioning frame, an LED epitaxial wafer and a second collimating lens, wherein the LED epitaxial wafer is provided in the positioning frame, and the second collimating lens is clamped in the positioning frame.

17. The light sterilizing apparatus according to claim 3, wherein:
the sterilizing light emitted by the light source mechanism is ultraviolet LED light with a wavelength of 200-280 nm, the light source mechanism comprises a positioning frame, an LED epitaxial wafer and a second collimating lens, wherein the LED epitaxial wafer is provided in the positioning frame, and the second collimating lens is clamped in the positioning frame.

18. The light sterilizing apparatus according to claim 4, wherein:
the sterilizing light emitted by the light source mechanism is ultraviolet LED light with a wavelength of 200-280 nm, the light source mechanism comprises a positioning frame, an LED epitaxial wafer and a second collimating lens, wherein the LED epitaxial wafer is provided in the positioning frame, and the second collimating lens is clamped in the positioning frame.

19. The light sterilizing apparatus according to claim 5, wherein:
the sterilizing light emitted by the light source mechanism is ultraviolet LED light with a wavelength of 200-280 nm, the light source mechanism comprises a positioning frame, an LED epitaxial wafer and a second collimating lens, wherein the LED epitaxial wafer is provided in the positioning frame, and the second collimating lens is clamped in the positioning frame.

20. The light sterilizing apparatus according to claim 6, wherein:
the sterilizing light emitted by the light source mechanism is ultraviolet LED light with a wavelength of 200-280 nm, the light source mechanism comprises a positioning frame, an LED epitaxial wafer and a second collimating lens, wherein the LED epitaxial wafer is provided in the positioning frame, and the second collimating lens is clamped in the positioning frame.
